(19) United States
(12) Patent Application Publication
(10) Pub. No.: US 2006/0206114 A1
(43) Pub. Date: Sep. 14, 2006

(54) Rod coupling assemblies

(75) Inventors: Michael D. Ensign, Salt Lake City, UT (US); David T. Hawkes, Pleasant Grove, UT (US); Thomas M. Sweeney II, Sarasota, FL (US)

Correspondence Address: RADER, FISHMAN & GRAUER PLLC, 10653 SOUTH RIVER FRONT PARKWAY, SUITE 150, SOUTH JORDAN, UT, 84095, US

(73) Assignee: Alphaspine, Inc., Sarasota, FL (US)

(21) Appl. No.: 11/284,438

(22) Filed: Nov. 21, 2005

Related U.S. Application Data
(60) Provisional application No. 60/629,840, filed on Nov. 19, 2004.
(60) Provisional application No. 60/666,819, filed on Mar. 30, 2005.
(60) Provisional application No. 60/672,590, filed on Apr. 18, 2005.
(60) Provisional application No. 60/703,622, filed on Jul. 29, 2005.
(60) Provisional application No. 60/703,684, filed on Jul. 29, 2005.

(51) Int. Cl. A61F 2/30; A61B 17/58

(52) U.S. Cl. 606/61; 606/72

(57) ABSTRACT

The disclosure generally relates to rod-coupling assemblies for coupling rods from separate pedicle screw systems. The rod-coupling assemblies generally include at least one rod-receiving body and at least one cam mechanism. The rod-receiving body and cam mechanism cooperate to capture and retain a distraction rod therewith. The cam mechanism includes a helical cam surface that mates with the rod and urges the rod into a retained position by rotation of the cam mechanism. In one embodiment, the assembly is configured to capture and retain two rods with one rod-receiving body. In another embodiment, the assembly is configured to capture and retain a rod and a head portion of a pedicle screw in one rod-receiving body. In yet another embodiment, the assembly includes a lever that is actuated to fix a first rod-receiving body to a second rod-receiving body.

### CROSS-REFERENCES TO RELATED APPLICATIONS

This application claims the benefit under 35 U.S.C. § 119(e) of U.S. Provisional Patent Application Nos. 60/629,840 filed Nov. 19, 2004; 60/666,819 filed Mar. 30, 2005; 60/672,590 filed Apr. 18, 2005; 60/703,622 filed Jul. 29, 2005; and 60/703,684 filed Jul. 29, 2005, where these provisional applications are incorporated herein by reference in their entireties.

### BACKGROUND OF THE INVENTION

1. Field of the Invention

The present invention relates generally to a rod-coupling assembly for receiving, capturing, and/or securing at least one distraction rod that is used in a spinal operation, for example in an operation to internally correct and/or structurally support a number of vertebral bodies.

2. Description of the Related Art

Various systems for internal fixation of bone segments in the human or animal body are known in the art. One type of system is a pedicle screw system, which is sometimes used as an adjunct to spinal fusion surgery, and which provides a means of gripping a spinal segment. A conventional pedicle screw system comprises a pedicle screw, two rod-receiving devices, and a rod (commonly referred to as a distraction rod) secured at one end by a first rod-receiving device and secured at a second end by a second rod-receiving device.

The pedicle screw includes an externally threaded stem and a head portion. The rod-receiving device couples to the head portion of the pedicle screw and receives the rod. For some surgeries, two such systems are inserted side-by-side into respective vertebrae and adjusted to distract and/or stabilize a spinal column, for instance during an operation to correct a herniated disk. The pedicle screw does not, by itself, fixate the spinal segment, but instead operates as an anchor point to receive the rod-receiving device, which in turn receives the rod. One goal of such a system is to substantially reduce and/or prevent relative motion between the spinal segments that are being fused.

### BRIEF SUMMARY OF THE INVENTION

The embodiments described herein are generally related to a bone fixation assembly that can be used to reinforce and/or augment a pedicle screw system for the internal fixation of vertebral bodies. The bone fixation assemblies described herein may be used to stiffen and strengthen a pedicle screw construct by joining one construct to another, to connect the rods of two pedicle screw systems to extend a fusion, for example during a re-operation or to transition rod sizes, or by providing one construct with another fixation point on the spine, for example using an extended hook to couple a rod to a posterior element of the spine. These bone fixation assemblies may be employed when minimally invasive surgery (MIS) techniques are used. The bone fixation assemblies described herein each generally include a rod-receiving body and a cam mechanism that operate together to capture and retain at least a distraction rod.

In one aspect, a rod-coupling assembly includes a rod-receiving body having a first channel positioned adjacent to a first opening. The first channel extends substantially parallel to a first wall and is configured to receive at least a portion of a distraction rod. In addition, the assembly includes a cam mechanism having a cam body with a contoured surface. The cam mechanism is rotatable in the first opening to urge the distraction rod into contact with a portion of the first wall.

In another aspect, a rod-coupling assembly includes a first rod-receiving body having a first channel positioned adjacent to a first opening. The first channel extends substantially parallel to a first wall and is configured to receive at least a portion of a first distraction rod. The assembly further includes a first cam mechanism having a first cam body with a contoured surface. The first cam mechanism is rotatable in the first opening to urge the first distraction rod into contact with a portion of the first wall. Further, the assembly includes a second rod-receiving body having a second channel, a second opening, and an elongated pocket. The second channel is positioned adjacent to the second opening, extends substantially parallel to a second wall, and is configured to receive at least a portion of a second distraction rod. The assembly also includes a pin, a lever, a second cam mechanism, and a transverse connector. The lever is received in the elongated pocket of the rod-receiving body and is rotatably coupled to the rod-receiving body via the pin. The second cam mechanism has a second cam body with a contoured surface. The second cam mechanism is rotatable in the second opening to urge the second distraction rod into contact with a portion of the lever. A transverse connector includes a first end, a second end, and an intermediate portion. The first end of the connector is coupled to the first rod-receiving body, the second end of the connector is received in the second rod-receiving body below the lever, and the intermediate portion is engageable with a portion of the lever.

In yet another aspect, a rod-coupling assembly includes a first rod-receiving body having a first channel positioned adjacent to a first opening. The first channel extends substantially parallel to a first wall and is configured to receive at least a portion of a first distraction rod. A first cam mechanism includes a first cam body with a contoured surface. The first cam mechanism is configured to be received in the first opening of the first rod-receiving body. A second rod-receiving body includes a second channel positioned adjacent to a second opening. The second channel extends substantially parallel to a second wall and is configured to receive at least a portion of a second distraction rod. In addition, a second cam mechanism includes a second cam body with a contoured surface. The second cam mechanism is configured to be received in the second opening of the second rod-receiving body. A connection rod extends between a first portion and a second portion, where the first portion includes an engagement surface to mate with the first cam mechanism and the second portion also includes an engagement surface to mate with the second cam mechanism. The first and second cam mechanisms are rotatable in the respective openings to secure the respective rods to the rod-coupling assembly.

In still yet another embodiment, a method of coupling a rod to a rod-receiving device includes receiving a rod in a channel of the rod-receiving device; and then rotating a cam mechanism by an amount sufficient to secure the rod between a helical cam surface of the cam mechanism and a portion of the rod-receiving device.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 is an isometric view of a rod-coupling assembly comprising a rod-receiving body and a cam mechanism, where the assembly is configured to secure two distraction rods therewith, according to one illustrated embodiment.

FIG. 2 is an exploded, isometric view of the rod-coupling assembly of FIG. 1.

FIG. 3 is cross-sectional view of the cam mechanism of FIG. 1, taken along line 3-3 of FIG. 2.

FIGS. 4A and 4B are isometric views of the rod-coupling assembly of FIG. 1 showing alternate ways of inserting the cam mechanism into the rod-receiving body.

FIG. 5 is an isometric view of another rod-coupling assembly where the rod receiving body is configured to receive rods having different diameters.

FIG. 6 is an exploded, isometric view of a rod-coupling assembly comprising a rod-receiving body and a cam mechanism, where the assembly is configured to secure a distraction rod and a head portion of a pedicle screw therewith, according to one illustrated embodiment.

FIGS. 7A-7E show various stages of securing the distraction rod and the head portion of the pedicle screw to the assembly of FIG. 6.

FIG. 8 is an isometric view of a rod-coupling assembly comprising a rod-receiving body, a cam mechanism, and an extension member, the assembly being configured to secure two distraction rods therewith, according to one illustrated embodiment.

FIG. 9 is an exploded, isometric view of the rod-coupling assembly of FIG. 8.

FIGS. 10A-10D show various stages of securing the distraction rod to the assembly of FIG. 8.

FIG. 11 is an isometric view of a rod-coupling assembly comprising a first rod-receiving body coupled to a second rod-receiving body with a transverse connector, according to one illustrated embodiment.

FIG. 12 is an exploded, isometric view of the rod-coupling assembly of FIG. 11.

FIG. 13 is a side elevational view of one of the cam mechanisms of FIG. 11.

FIG. 14 is a cross-sectional view of the assembly of FIG. 11, taken along line 14-14 of FIG. 11.

FIG. 15 is an isometric view of a rod-coupling assembly comprising two rod-receiving bodies coupled together via a connector, according to one illustrated embodiment.

FIG. 16 is a schematic view showing a relative position of the connector of FIG. 15 in view of two distraction rods that are received by the rod-receiving bodies.

### DETAILED DESCRIPTION OF THE INVENTION

In one embodiment, pedicle screw systems may be fixed in the spine, for example to perform spinal fixation and/or corrective surgeries, which surgeries may be performed via minimally invasive surgery (MIS) techniques. The systems are inserted into the pedicles of the spine and then interconnected with rods to manipulate (e.g., correct the curvature, compress or expand, and/or structurally augment) at least portions of the spine. Using the MIS approach to spinal fixation and/or correction surgery has been shown to decrease a patient's recovery time and reduce the risks of follow-up surgeries.

The ability to efficiently fix and/or correct a spine during surgery often necessitates that the pedicle screw systems be reinforced and/or augmented to achieve better stiffness and strength. Such reinforcement systems, once installed, permit the entire installation to be more robust when influenced by dynamic and static loads. In addition, the reinforcement assemblies permit at least two pedicle screw systems to work in tandem, which allows for equitable and efficient load distribution. For example, if a first pedicle screw system is placed on softer bone while an adjacent system is placed in healthier bone and then the systems are connected by a reinforcement assembly, the system in the healthier bone can take more of the operation forces and in turn reduce some of the stress from the other system (i.e., due to the relative stiffnesses of the healthier versus softer bone). Such an installation may be advantageous in allowing the softer bone to heal and harden quicker without being overly stressed in the interim.

The term “distraction,” when used in a medical sense generally relates to joint surfaces and suggests that the joint surfaces move perpendicular to one another. However when “traction” and/or “distraction” is performed, for example on spinal sections, the spinal sections may move relative to one another through a combination of distraction and gliding.

Rod-to-Rod Coupling Assembly

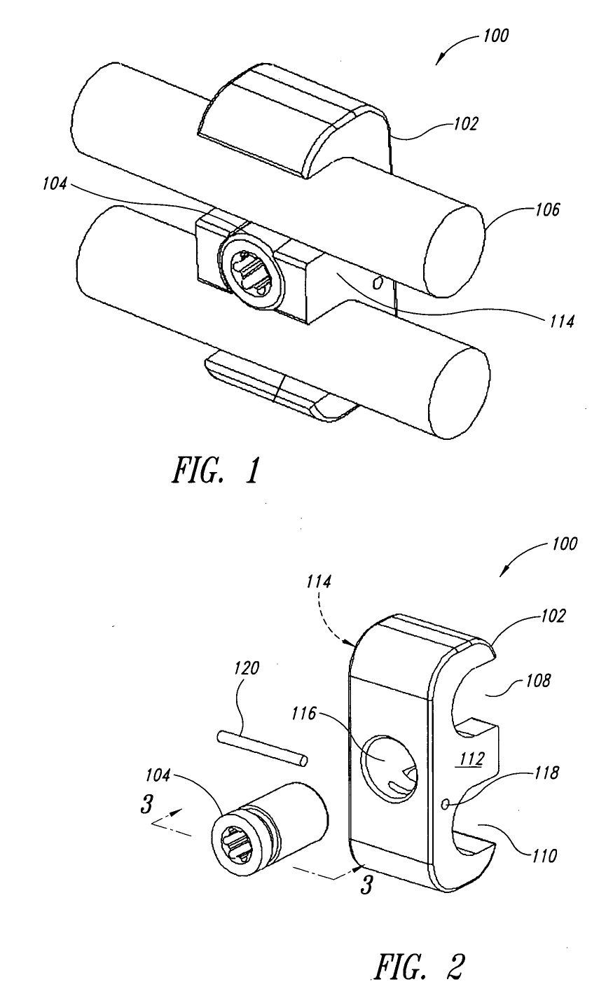

FIG. 1 generally shows a rod-coupling assembly 100 comprising a rod-receiving body 102 and cam mechanism 104. The rod-receiving body 102 is configured to receive two distraction rods 106, according to the illustrated embodiment. The cam mechanism 104 is a rotatable cam screw that operates with the rod-receiving body 102 to lock or unlock the rods 106 in situ. One purpose of the rod-coupling assembly 100 is to connect or join two pedicle screw systems.

FIG. 2 shows the components of the rod-coupling assembly 100. The rod-receiving body 102 includes a first rod slot or channel 108 and a second rod slot or channel 110. Each rod slot 108, 110 is configured as a U-shaped channel extending from a first surface 112 to a second surface 114 of the body 102, according to the illustrated embodiment. The U-shaped channels 108, 110 are sized to receive the distraction rods 106. The respective rod slots 108, 110 may receive rods 106 of different diameters, as will be described in more detail below.

The rod-receiving body 102 further includes a first opening 116 and a second opening 118. The first opening 116 is configured to receive the cam mechanism 104 and is located between the first and second rod slots 108, 110. The second opening 118 extends longitudinally from the first surface 112 to the second surface 114 and is configured to receive a retaining pin 120.

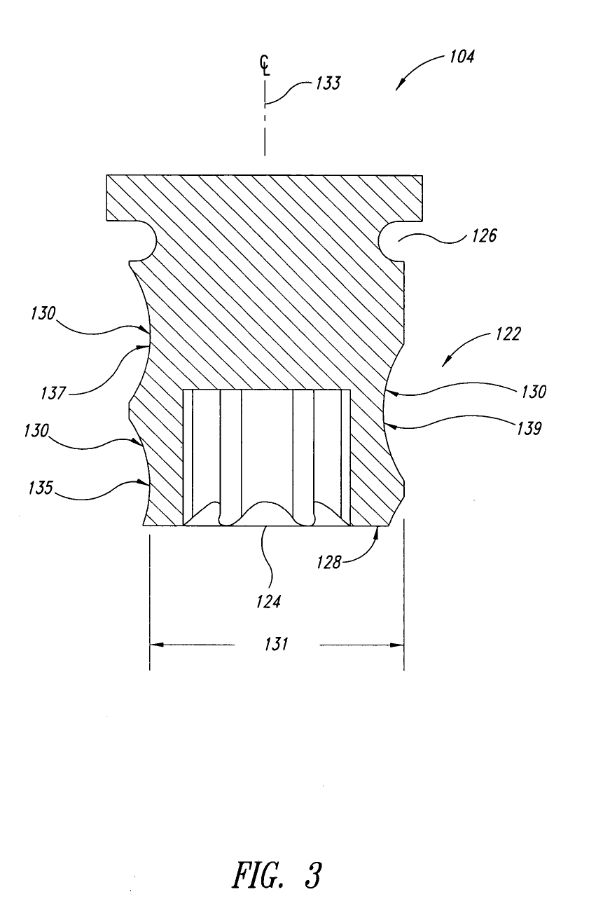

FIG. 3 shows a cross-sectional view of the cam mechanism 104 comprising a cam body 122, a tool engagement portion 124, a groove 126, and an end surface 128. The body 122 includes a helical cam surface 130. In one embodiment, an effective diameter 131 of the helical cam surface 130 can vary along the longitudinal length of the cam body 122. The effective diameter 131 is taken with respect to a centerline and/or rotational axis 133 of the cam mechanism 104. The helical cam surface 130 of the cam mechanism 104 is contoured to cooperate with the diameter of the rod 106. The helical cam surface 130 includes a lead-in portion 135 and then tapers and/or runs out as the helical cam surface 130 winds around the cam body 122 toward the groove 126.

The tool engagement portion 124 is configured to receive a castellated torque device, according to the illustrated embodiment. It is understood, however, that the tool-engagement portion 124 may be configured to receive a variety of tools, such as a flat head screwdriver, a Philips head screwdriver, a hexagonal ratchet head, or some other type of tool capable of rotating the cam mechanism 104. The groove 126 is an arcuate and/or convex detent formed circumferentially around the body 122 and sized to be about the same diameter or larger than the diameter of the retaining pin 120. The groove 126 may have a shape other than an arc and/or convex shape, for example the groove 126 may be square or elliptical to receive a like retaining pin 120. The retaining pin 120 operates to transitionally couple the cam mechanism 104 to the rod-receiving body 102, while allowing the cam mechanism 104 to be rotated relative to the rod-receiving body 102. Alternatively stated, the insertion of the retaining pin 120 into the rod-receiving body 102 and the groove 126 of the cam mechanism 104 keeps the cam mechanism 104 from sliding out of the first opening 116 of the rod-receiving body 102.

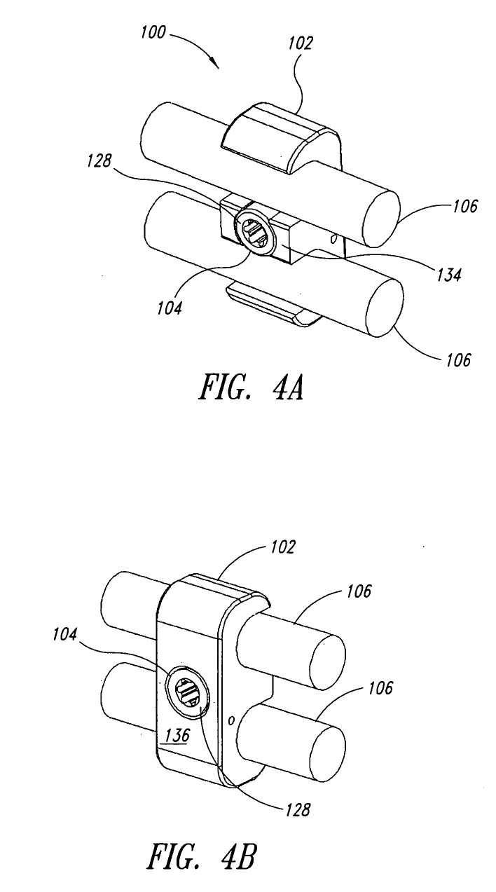

FIGS. 4A and 4B show various embodiments of the rod-coupling assembly 100. FIG. 4A shows the cam mechanism 104 inserted into the rod-receiving body 102 such that the end surface 128 of the cam mechanism 104 is approximately flush with a bottom surface 134 of the rod-receiving body 102. FIG. 4B shows the cam mechanism 104 inserted into the rod-receiving body 102 in an opposite manner such that the end surface 128 of the cam mechanism 104 is approximately flush with a top surface 136 of the rod-receiving body 102.

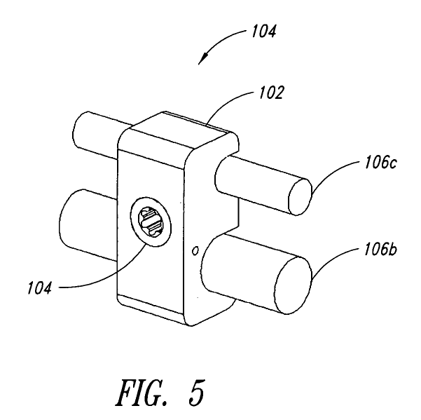

FIG. 5 shows the rod-coupling body 102 having different sized first and second slots 108, 110, respectively, to receive two different diameter distraction rods 106a, 106b.

During installation and after the pin 120 has been inserted to retain the cam mechanism 104 in the first opening 116 of the rod-receiving body 102, the cam mechanism 104 is rotated to capture and retain the distraction rods 106. In one embodiment, one distraction rod 106 is captured and retained between a first portion 137 (FIG. 3) of the helical cam surface 130 of the cam body 122 and the rod-receiving body 102, while another distraction rod 106 is captured and retained (i.e., secured) between a second portion 139 (FIG. 3) of the helical cam surface 130 of the cam body 122 and the rod-receiving body 102. It is understood that the rods 106 will be captured and retained at offset positions relative to the depth of the rod-receiving body 102. Referring to FIG. 3, it is shown that the surfaces 137, 139, which are in contact with the respective rods 106, are respectively offset by one-half pitch of the cam mechanism 104. The helical cam surface 130 of the cam mechanism 104 is configured to urge the rod 106 one pitch for every full rotation (i.e., 360 degrees) of the cam mechanism 104.

Rod-to-Screw Coupling Assembly

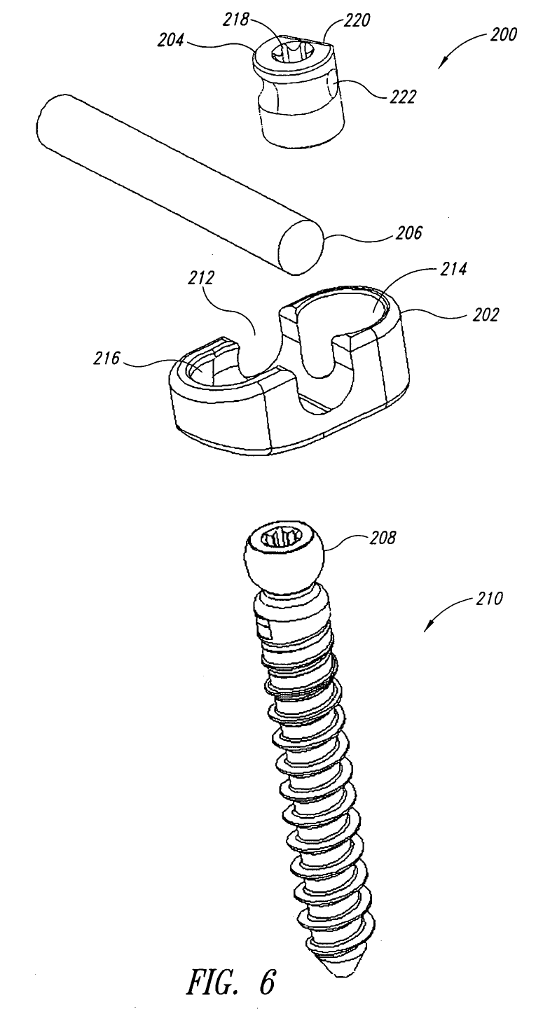

FIG. 6 shows another rod-coupling assembly 200 comprising a rod-receiving body 202 and a cam mechanism 204. The rod-receiving body 202 is configured to receive a distraction rod 206 and a head portion 208 of a pedicle screw 210, according to the illustrated embodiment. The cam mechanism 204 is a rotatable cam screw that operates with the rod-receiving body 102 to lock or unlock the rod 106 and the head portion 208 of the pedicle screw 210 with respect to the rod-receiving body 202. One purpose of the rod-coupling assembly 200 is to offset the rod 206 from the pedicle screw 210 because of spatial constraints within the surgery site, for example.

The rod-receiving body 202 includes a rod slot 212, a first opening 214, and a second opening 216. The rod slot 212 is a U-shaped channel. The first opening 214 is configured to receive the cam mechanism 204, while the second opening 216 is configured to receive the head portion 208 of the pedicle screw 210. The rod slot 212 is located between the first opening 214 and the second opening 216.

The cam mechanism 204 includes a tool engagement portion 218, a truncated portion 220, and a helical cam surface 222. The tool engagement portion 218 is similar to the tool engagement portion 124 of the previous embodiment. The operation of the cam mechanism 204, in particular the function of the truncated portion 220 and the helical cam surface 222, is described below. In all other respects, the rod-coupling assembly 200 is generally structurally and functionally similar to the previously described embodiment.

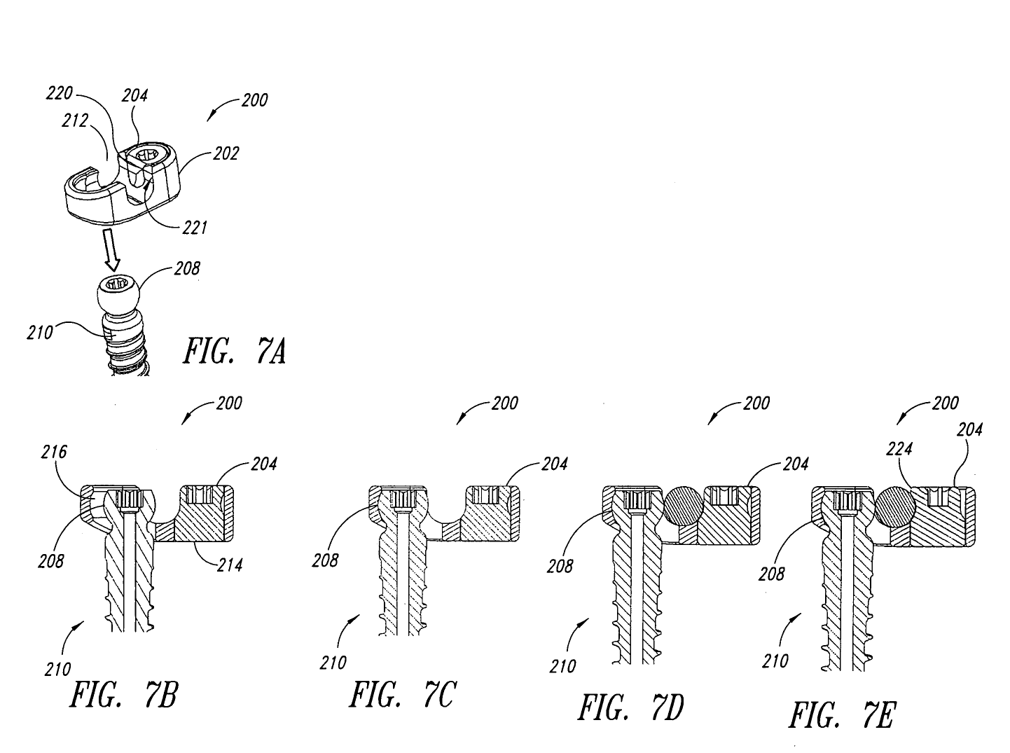

FIGS. 7A-7E show various stages of the rod-coupling assembly 200 receiving, and subsequently securing the head portion 208 of the pedicle screw 210 and the rod 206 to the rod-coupling assembly 200. FIG. 7A shows the rod-receiving body 202 being placed onto the head portion 208 of the pedicle screw 210. The head portion 208 is received through the second opening 216. The cam mechanism 204 can be pre-assembled with and located in the first opening 214 of the rod-receiving body 202. Alternatively, the cam mechanism 204 can be placed in the first opening 214 of the rod-receiving body 202 intra-operatively (i.e., during surgery). The cam mechanism 204 is positioned in the rod-receiving body 202 such that the truncated portion, which comprises a substantially flat surface 220, is adjacent to and substantially aligned with a first surface 221 of the rod-receiving body 202. Accordingly, the cam mechanism 204 is in an open position.

FIG. 7B shows the head portion 208 of the pedicle screw 210 being initially received in the second opening 216 of the rod-receiving body 202. FIG. 7C shows the head portion 208 of the pedicle screw 210 being seatably engaged with rod-receiving body 202. In one exemplary embodiment, the pedicle screw 210 is inserted into the spinal bone, so that seatably engaging the head portion 208 with the rod-receiving body 202 includes moving the body 202 laterally with respect to the pedicle screw 210 that is fixed in the bone. The second opening 216 of the body 202 is elongated to permit lateral, relative motion between the body 202 and the pedicle screw 210. Moving the rod-receiving body 202 relative to the head portion 208 can be achieved by using an instrument or tool or by manually manipulating the body 202 and/or the screw 210.

FIG. 7D shows the rod 206 being initially inserted into the rod slot 212 of the rod-receiving body 202 with the cam mechanism 204 still in the open position. FIG. 7E shows the cam mechanism 204 rotated to a closed position where the helical cam surface 222 operates to capture the rod 206 and urge the rod 206 against the head portion 208 of the pedicle screw 210. In turn, the head portion 208 is urged against the rod-receiving body 202. Thus, the rotation of the cam mechanism 204 causes both the rod 206 and the head portion 208 of the pedicle screw 210 to be secured in the rod-coupling assembly 200. The cam mechanism 204 may include a lip 224 to help capture and secure the rod 206. Further, a spacer can be placed between the rod 206 and the head portion 208 of the pedicle screw 210 to provide variable amounts of lateral offset.

Device for Coupling to a Vertebral Body

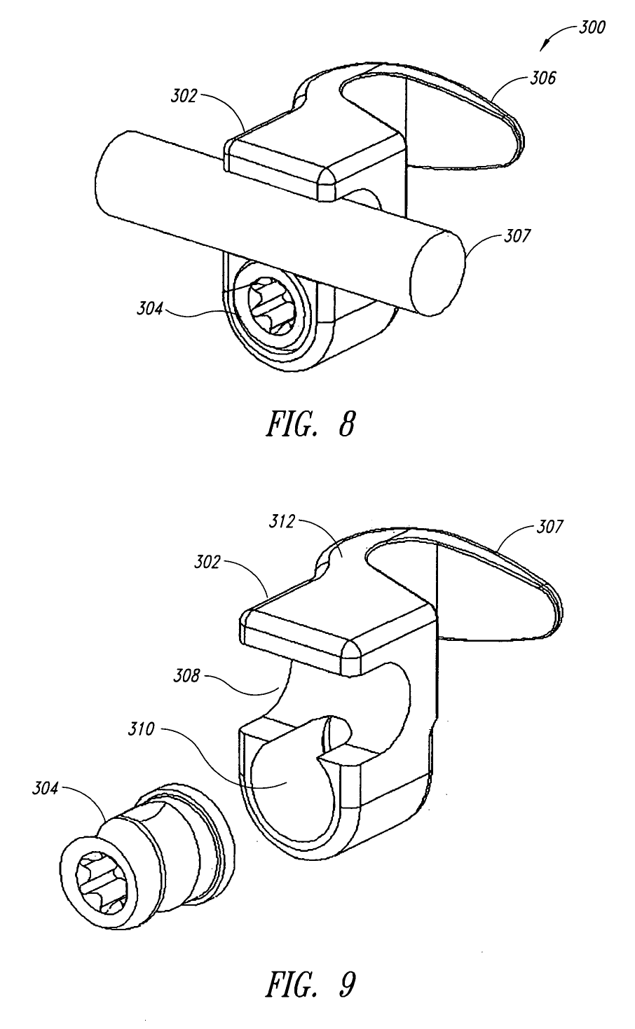

FIG. 8 shows another rod-coupling assembly 300 having a rod-receiving body 302, a cam mechanism 304, and an extension member 306. In one embodiment, the rod-coupling assembly 300 is used to connect a rod to a posterior element of the spine and can operate with other rod-coupling assemblies and distractions rods 307 to induce an amount of spinal correction in a patient.

FIG. 9 shows the rod-receiving body 302 having a rod slot 308 and a first opening 310. The rod slot 308 is a U-shaped channel while the first opening 310 is configured to receive the cam mechanism 304. The extension member 306 extends from a portion 312 of the rod-receiving body 302. The extension member 306 can be integrally formed with the body 302 or may be mechanically fixed thereto. The cam mechanism 304 is substantially similar to the cam mechanism 104, which is described above in reference to FIG. 3; therefore the cam mechanism 304 will not be described in further detail. The cam mechanism 304 can be transitionally fixed to the rod-receiving body 302 with a pin (not shown) in a manner similar to that described above and illustrated in FIG. 2.

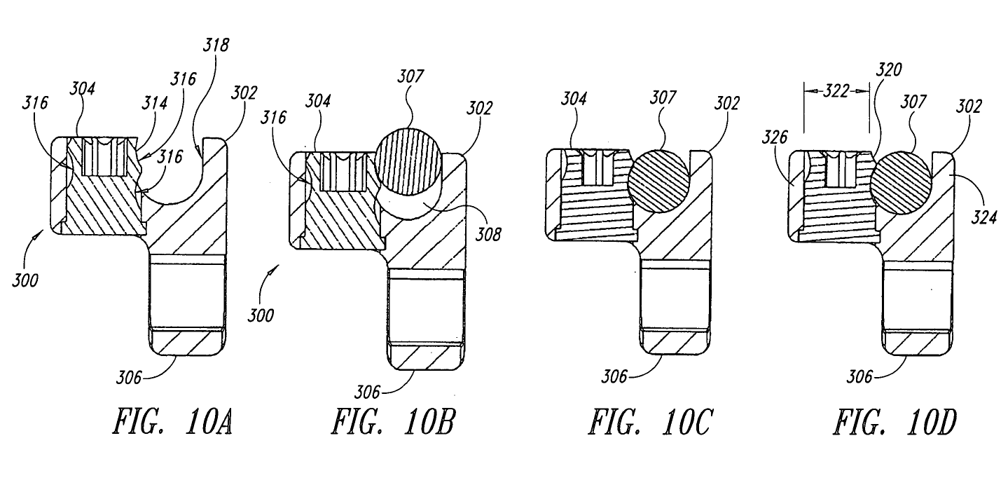

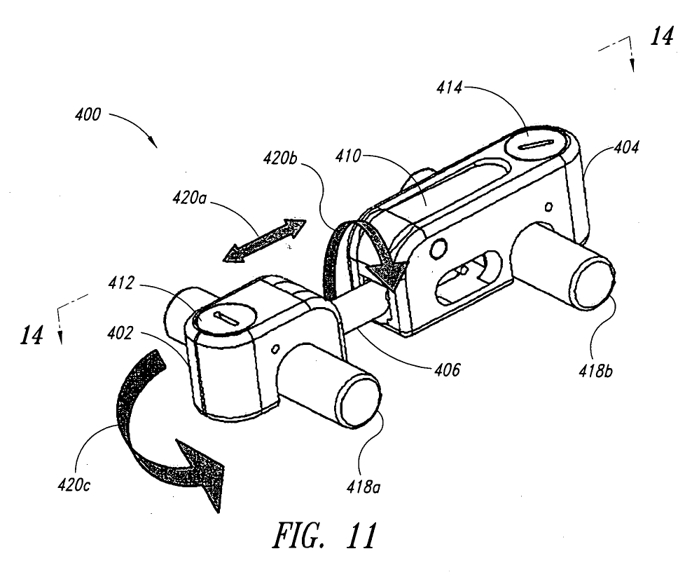

FIGS. 10A-10D show various stages of the rod 307 being placed, captured, and then secured by the rod-coupling assembly 300. FIG. 10A shows the cam mechanism 304 pre-assembled with the rod-receiving body 302, where the cam mechanism 304 is received in the first opening 310 of the body 302. The cam mechanism 304 is rotationally positioned in a rod-receiving position such that a first portion 314 of a helical cam surface 316 faces the rod slot 308.

FIG. 10B shows the rod 307 initially placed into the rod-coupling assembly 300. The first portion 314 of the helical cam surface 316 and a far surface 318 of the rod-receiving body 302 initially support the rod 307, where the far surface 318 defines one side or wall of the rod slot 308.

FIG. 10C shows the rod 307 being urged further into the rod slot 308 due to the rotational action of the cam mechanism 304, where the threaded cam surface 316 directs the rod 307 into the rod slot 308.

FIG. 10D shows the rod 307 secured in the rod slot 308. In one embodiment, the rod 307 is captured in the rod slot 308 by the lip 320, which prevents the rod 307 from escaping from the rod slot 308. Additionally or alternatively, the rod 307 is captured due to an effective diameter 322 of the cam mechanism 304 creating a compressive force on the rod 307 to frictionally secure the rod 307 between the cam mechanism 304 and the surface 318.

First Transverse Connector

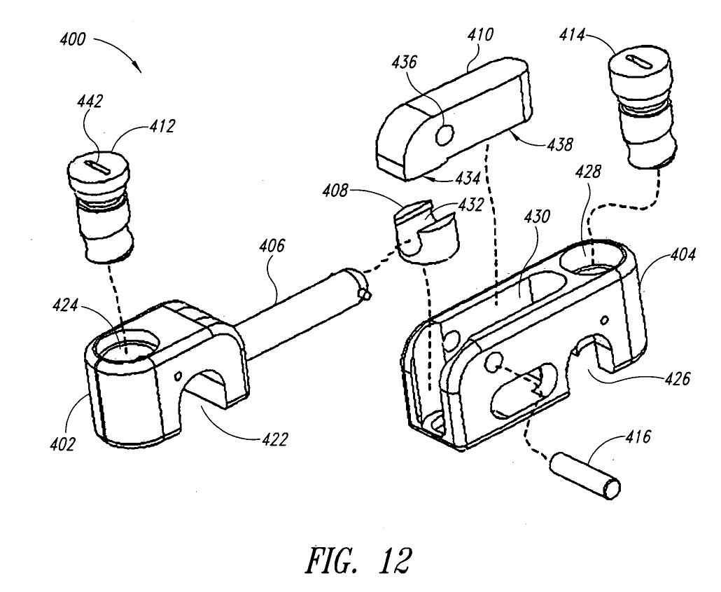

FIGS. 11 and 12 show a rod-coupling assembly 400 having a first rod-receiving body 402, a second rod-receiving body 404, a transverse connector 406, a seat 408, a lever 410, first and second cam mechanisms 412, 414, and a retainer pin 416. In one embodiment, the rod-coupling assembly 400 is used to rigidly couple two spinal distraction rods 418a, 418b. This rod-coupling assembly 400 uses only using two cam mechanisms 412, 414, which provides an advantage over conventional systems that utilize at least three, sometimes four, fastener elements to lock down the various components of the conventional systems. In addition, having to manipulate three or four fastener elements using MIS procedures can be more difficult and time consuming. When a cannula is used, for example, it is often difficult to access all three or four fastener elements through the cannula.

FIG. 11 shows that the rod-coupling assembly 400 adjusts to the rods 418a, 418b via three degrees of freedom as indicated by the arrows 420a, 420b, and 420c. The rod-coupling assembly 400 advantageously captures and locks the rods 418a, 418b with only two actions: (1) rotating the first cam mechanism 412, and then (2) rotating the second cam mechanism 414. It is understood that the aforementioned cam rotations can be done in reverse order where the second cam mechanism 414 is rotated first.

FIG. 12 shows the various components of the rod-coupling assembly 400 separated from one another, according to one illustrated embodiment. The first rod-receiving body 402 includes a rod slot 422 and an opening 424 for receiving the cam mechanism 412. The second rod-receiving body 404 includes rod slot 426, a first opening 428 for receiving the cam mechanism 414, and a second, elongated pocket 430 for receiving the seat 408 and the lever 410.

The transverse connector 406 is coupled to and extends from the first rod-receiving body 402. In one embodiment, the transverse connector 406 is integrally formed with the first rod-receiving body 402 in that the body 402 and connector 406 comprise a monolithic part. The seat 408 includes a channel 432 configured to receive and support the transverse connector 406. In the illustrated embodiment, the channel 432 is U-shaped, however it is understood that the channel 432 and/or transverse connector 406 may have alternative configurations.

The lever 410 includes a first contact surface 434, a fulcrum point 436, and a second contact surface 438. The fulcrum point 436 is located between the first and second contact surfaces 434, 438, respectively. The pin 416 is used to couple the lever 410 with the second rod-receiving body 404, where the lever 416 is free to rotate relative to the second rod-receiving body 404.

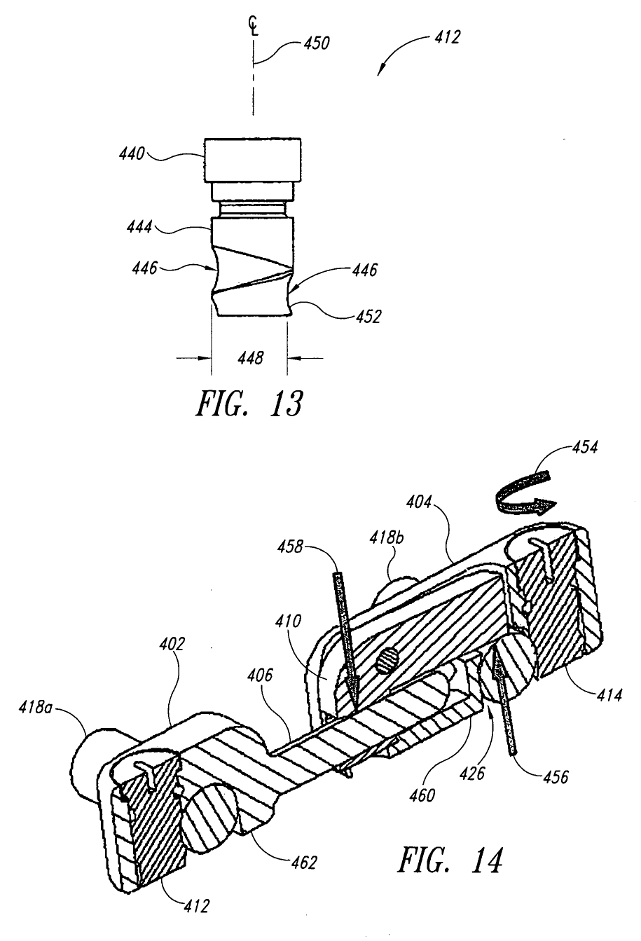

FIG. 13 shows one of the cam mechanisms 412, 414. For the sake of brevity and not being duplicative, only cam mechanism 412 is described in detail herein. The described structural and/or functional aspects are equally applicable to the cam mechanism 414, unless noted otherwise. The cam mechanism 412 includes a top portion 440 having an engagement portion 442 (FIG. 12). A cam body 444 extends from the top portion 440 and includes a helical cam surface 446. An effective diameter 448 of the helical surface 446 can vary along the longitudinal length of the cam body 444. The effective diameter 448 is taken with respect to a centerline line and/or rotational axis 450 of the cam mechanism 412. The helical cam surface 446 of the cam mechanism 412 cooperates with the diameter of the rod 418a. The helical cam surface 446 tapers and runs out as the helical cam surface 446 winds around the cam body 444 toward the top portion 440. The effective diameter of the run-out portion of the helical cam surface 446 is sized to forcibly retain the rod 418a in the assembly 400, as described in detail below.

FIG. 14 shows a cut-away view of the rod-coupling assembly 400, which is pre-assembled and then placed on the rods 418a, 418b during a spinal operation, according to the illustrated embodiment. The first rod-receiving body 402 and the transverse connector 406 are slidably adjustable (e.g., arrow 420a in FIG. 11) relative to the second rod-receiving body 404 so that the respective bodies 402, 404 can be moved relative to one another to account for an amount of separation of the rods 418a, 418b. The rods 418a, 418b are initially placed in contact with first portions 452 (FIG. 13) of the cam mechanisms 412, 414.

The cam mechanism 414 is rotated by a first amount as indicated by arrow 454. This rotation 454 urges the rod 418b further into the rod slot 426 of the second rod-receiving body 404. The rod 418b contacts the second contact surface 438 of the lever 410, as indicated by the arrow 456. This contact force 456 causes the lever 410 to rotate about the fulcrum point 436 (i.e., pin 416). This rotation of the lever 410 results in an applied force 458 on the transverse connector 406, where the first contact surface 434 of the lever 410 contacts the transverse connector 406. The applied force 458 causing the contact between the first contact surface 434 of the lever 410 and the transverse connector 406 laterally fixes the first rod-receiving body 402 to the second rod-receiving body 404. Alternatively stated, the rotation of the lever 410 constrains the translational degree of freedom 420a, which is identified in FIG. 11.

The rotation 454 of the cam mechanism 414 further captures and secures the rod 418b with the assembly 400. The helical cam surface 446 of the cam body 444 acts to frictionally urge and retain the rod 418b between the cam mechanism 414 and a portion 460 of the second rod-receiving body 404. In a similar manner, rotation of the cam mechanism 412 in the first opening 424 of the first rod-receiving body 402 captures and secures the rod 418a between the cam mechanism 412 and a portion 462 of the first rod-receiving body 402. In one embodiment, the rotation of the cam mechanism 412 does not impart any stress to the transverse connector 406, lever 410, and/or second rod-receiving body 404.

Second Transverse Connector

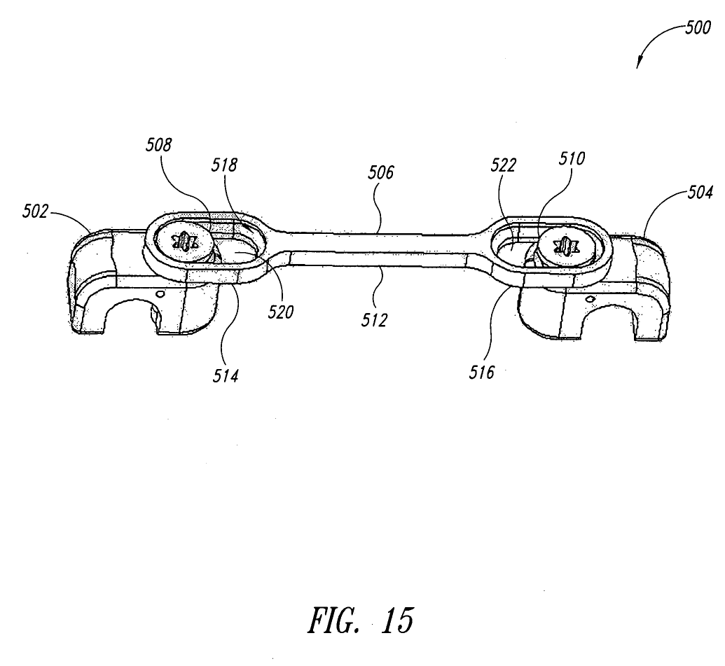

FIG. 15 shows another rod-coupling assembly 500 having a first rod-receiving body 502, a second rod-receiving body 504, a connector 506, and respective first and second cam mechanisms 508, 510. The first and second rod-receiving bodies 502, 504 are structurally and functionally similar to at least one of the aforementioned embodiments, therefore these components will not be described in further detail with respect to the illustrated embodiment. Likewise, the cam mechanisms 508, 510 are structurally and functionally similar to at least one of the aforementioned embodiments, thus the cam mechanism 508, 510 will not be described in any further detail. One purpose of the rod-coupling assembly 500 is to provide a quick means to connect or join two pedicle screw systems.

The connector 506 includes a two-force rod member 512 coupled between first and second end portions 514, 516. The first end portion 514, which is representative of the second end portion 516, includes an inner surface 518 that encompasses an oval or elliptical shaped opening 520, according to the illustrated embodiment. The inner surface 518 is contoured to mate with a portion of a helical cam surface (see FIG. 3) of the cam mechanism 508.

During installation, the first cam mechanism 508 is placed into the first rod-receiving body 502 through the opening 520 of the first end portion 514. Rotation of the first cam mechanism 508 permits the threaded helical cam surface of the cam mechanism 508 to work in cooperation with the first rod-receiving body 502 to capture and secure a first rod (not shown). Likewise, a second portion of the threaded helical cam surface engageably mates with the inner surface 518 of the first end portion 514 of the connector 506.

In a similar fashion, the second cam mechanism 510 is placed into the second rod-receiving body 504 through an opening 522 in the second end portion 516 of the connector 506. Rotation of the second mechanism 510 results in capturing and securing a second rod (not shown). This rotation further results in the engagement of the second cam mechanism 510 with the second end portion 516 of the connector 506.

The rod-coupling assembly 500 advantageously permits two rods to be quickly and easily coupled together. In addition, the rod-coupling assembly 500 has a low profile, which tends to minimize the impact and trauma on the tissue in the vicinity of the vertebral body.

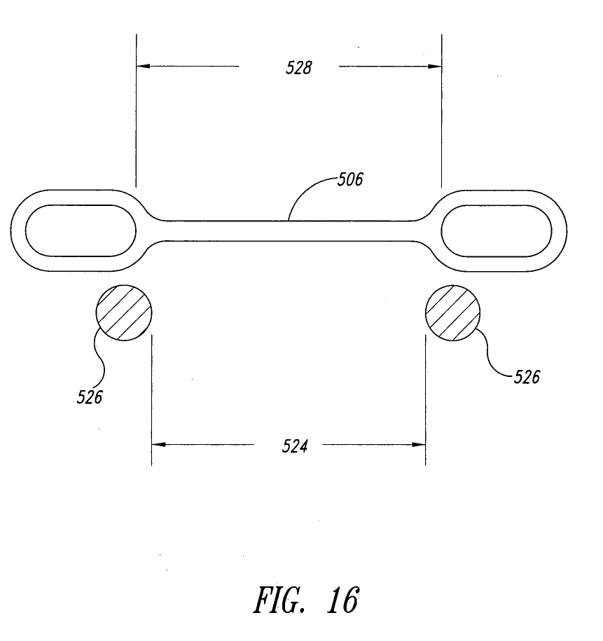

FIG. 16 schematically shows that the connector 506 operates to couple the first and second rod-receiving bodies 502, 504 together and to place the rod member 512 in compression, according to one embodiment. Compression of the rod member 512 occurs when the distance 524 between the rods 526 is less than an operative length 528 of the connector 506. Hence, the installment of the connector 506 tends to force the rods 526 apart. This is an installation method that can be used to separate a herniated disc, for example.

The amount of compression can be adjusted by utilizing different length connectors 506. The induced residual compression (i.e., preload) in the assembly 500 may advantageously permit the assembly 500 to be more resistant to fatigue damage and/or failure. Because any applied tensile stress (e.g., bending, pulling, etc.) must first exceed the amount of residual compressive stress already present in the assembly 500, the net amount of potentially damaging tensile stress can be reduced, which means that fatigue damage in the assembly 500 may not accumulate as fast, thus resulting in a longer operational life of the assembly 500. In turn, the longer operational life may reduce a number of downstream surgeries to repair, correct, and/or re-align any internal spinal correction hardware. These advantages, as well as other advantages, will be apparent to those skilled in the art and are applicable to the present embodiment and other embodiments, or combinations thereof, described herein.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to various screws and rods, not necessarily the exemplary pedicle screws and distraction rods generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, to include U.S. Provisional Patent Application Nos. 60/629,840 filed Nov. 19, 2004; 60/666,819 filed Mar. 30, 2005; 60/672,590 filed Apr. 18, 2005; 60/703,622 filed Jul. 29, 2005; and 60/703,684 filed Jul. 29, 2005, are incorporated herein by reference, in their entirety. Aspects of the invention can be modified, if necessary, to employ screws, materials and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all spine anchoring devices that operated in accordance with the claims. Accordingly, the invention is not limited by the disclosure, but instead its scope is to be determined entirely by the following claims.

1. A rod-coupling assembly comprising:
a rod-receiving body having a first channel positioned adjacent to a first opening, the first channel forming a first wall and configured to receive at least a portion of a distraction rod; and
a cam mechanism having a cam body with a contoured surface, the cam mechanism rotatable in the first opening to cooperate with a portion of the first wall to couple the distraction rod to the rod-receiving body.

2. The rod-coupling assembly of claim 1 wherein the first channel is U-shaped and extends from a first exterior surface to a second exterior surface of the body.

3. The rod-coupling assembly of claim 1, further comprising:
a hook device coupled to and extending from a bottom portion of the body.

4. The rod-coupling assembly of claim 3 wherein the hook device is integrally coupled to the bottom portion of the body.

5. The rod-coupling assembly of claim 1 wherein the cam body includes a first portion, a second portion, and a rotational axis, wherein the first portion is positioned farther from the rotational axis than the second portion.

6. The rod-coupling assembly of claim 1 wherein the contoured surface of the cam body is a helical cam surface.

7. The rod-coupling assembly of claim 1 wherein the cam mechanism is a cam screw.

8. The rod-coupling assembly of claim 1 wherein the rod-receiving body includes a passageway that intersects the first opening.

9. The rod-coupling assembly of claim 8, further comprising:
a pin insertable into the passageway to translationally constrain the cam mechanism relative to the body.

10. The rod-coupling assembly of claim 1 wherein the rod-receiving body includes a second channel positioned substantially parallel to the first channel and wherein the first opening is located between the first channel and the second channel.

11. The rod-coupling assembly of claim 10 wherein the second channel is U-shaped and extends from a first exterior surface to a second exterior surface of the body.

12. The rod-coupling assembly of claim 10 wherein the cam body includes a first portion, a second portion, and a rotational axis, the first portion located at a first distance from the rotational axis, the second portion located at a second distance from the rotational axis.

13. The rod-coupling assembly of claim 12 wherein the first portion of the cam body cooperates with the rod-receiving body to capture and retain the distraction rods when the cam mechanism is rotated in the first opening.

14. The rod-coupling assembly of claim 1 wherein the rod-receiving body includes a second opening configured to receive a head portion of a pedicle screw.

15. The rod-coupling assembly of claim 14 wherein the first channel is located between the first opening and the second opening.

16. The rod-coupling assembly of claim 14 wherein rotation of the cam mechanism in the first opening urges the distraction rod into contact with the head portion of the pedicle screw to couple the distraction rod to the rod-receiving body.

17. A rod-coupling assembly comprising:
a first rod-receiving body having a first channel positioned adjacent to a first opening, the first channel forming a first wall and configured to receive at least a portion of a first distraction rod;
a first cam mechanism having a first cam body with a contoured surface, the first cam mechanism rotatable in the first opening to urge the first distraction rod into contact with of the first wall;
a second rod-receiving body having a second channel, a second opening, and an elongated pocket, the second channel positioned adjacent to the second opening, forming a second wall, and configured to receive at least a portion of a second distraction rod;
a pin;
a lever received in the elongated pocket of the rod-receiving body and rotatably coupled to the rod-receiving body via the pin;
a second cam mechanism having a second cam body with a contoured surface, the second cam mechanism rotatable in the second opening to urge the second distraction rod into contact with a portion of the lever; and
a transverse connector having a first end, a second end, and an intermediate portion, the first end coupled to the first rod-receiving body, the second end received in the second rod-receiving body, and the intermediate portion engageable with the lever.

18. The rod-coupling assembly of claim 17 wherein the first distraction rod is of a larger diameter than the second distraction rod.

19. The rod-coupling assembly of claim 17 wherein the first rod-receiving body is translationally adjustable relative to the second rod-receiving body.

20. The rod-coupling assembly of claim 17 wherein rotation of the second cam mechanism couples the transverse connector to the second rod-receiving body due to an amount of pressure exerted by the lever on the transverse connector.

21. A rod-coupling assembly comprising:
a first rod-receiving body having a first channel positioned adjacent to a first opening, the first channel forming a first wall and configured to receive at least a portion of a first distraction rod;
a first cam mechanism having a first cam body with a contoured surface, the first cam mechanism configured to be received in the first opening of the first rod-receiving body;
a second rod-receiving body having a second channel positioned adjacent to a second opening, the second channel extending substantially parallel to a second wall and configured to receive at least a portion of a second distraction rod;
a second cam mechanism having a second cam body with a contoured surface, the second cam mechanism configured to be received in the second opening of the second rod-receiving body; and
a connection rod extending between a first portion and a second portion, the first portion having an engagement surface to mate with the first cam mechanism, the second portion having an engagement surface to mate with the second cam mechanism, wherein the first and second cam mechanisms are rotatable in the respective openings to secure the respective rods to the rod-coupling assembly.

22. The rod-coupling assembly of claim 21 wherein the contoured surface of the first cam body includes a helical cam surface.

23. The rod-coupling assembly of claim 21 wherein the contoured surface of the second cam body includes a helical cam surface.

24. The rod-coupling assembly of claim 21 wherein the connection rod is a two-force member.

25. The rod-coupling assembly of claim 21 wherein the respective engagement surfaces of the connection rod are protuberances complementarily shaped to mate with helical cam surfaces of the respective cam mechanisms.

26. A method of coupling a rod to a rod-receiving device, the method comprising:
receiving a rod in a channel of the rod-receiving device; and
rotating a cam mechanism by an amount sufficient to secure the rod between a helical cam surface of the cam mechanism and a portion of the rod-receiving device.

27. The method of claim 26 wherein receiving the rod includes moving the rod-receiving device onto a portion of the rod.

28. The method of claim 26 wherein receiving the rod includes engaging the rod with a lead-in portion of the helical cam surface of the cam mechanism.

29. The method of claim 26 wherein rotating the cam mechanism includes engaging a portion of the cam mechanism with a tool.

30. The method of claim 26, further comprising:
receiving a head portion of a pedicle screw in an opening of the rod-receiving device and wherein rotating the cam mechanism to secure the rod and the head portion of the pedicle screw between respective portions of the helical cam surface of the cam mechanism and the rod-receiving device.

31. The method of claim 26, further comprising:
engaging a portion of a spine with a member extending from the rod-receiving device.
